SEQUENCE LISTING (6 sequences)

SEQ ID NO: 1 - protein, 1212 aa, Homo sapiens
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPAFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 2 - DNA, 4098 bp, Homo sapiens
ggtggcctctgtggccgtccaggctagcggcggcccgcaggcggcggggagaaagactctctcacctggtcttgcggctgtggccaccgccggccaggggtgtggagggcgtgctgccggagacgtccgccgggctctgcagttccgccgggggtcgggcagctatggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccagctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagttgaggaagaggatggcaagactgcaactcaaccactgttgaaaaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataaatgttctatacagtggacagccctccagaatggtacttcagtgcctagtgtagtaacctgaaatcttcaatgacacattaacatcacaatggcgaatggtgacttttctttcacgatttcattaatttgaaagcacacaggaaagcttgctccattgataacgtgtatggagacttcggttttagtcaattccatatctcaatcttaatggtgattcttctctgttgaactgaagtttgtgagagtagttttcctttgctacttgaatagcaataaaagcgtgttaactttttgg

SEQ ID NO: 3 - DNA, 22 bp, Artificial sequence
  Primer
cacctactacctgcgcaccttc

SEQ ID NO: 4 - DNA, 22 bp, Artificial sequence
  Primer
gaccacagcatctctggttgga

SEQ ID NO: 5 - protein, 12 aa, Artificial sequence
  synthetic peptide
SKKPKGFFGYKC

SEQ ID NO: 6 - protein, 16 aa, Artificial sequence
  synthetic peptide
SGESEPAKGSEEAKGC

(19) United States
(12) Patent Application Publication
(10) Pub. No.: US 2006/0088537 A1
(43) Pub. Date: Apr. 27, 2006

(54) Protein involved in cancer

(76) Inventor: Jonathan Alexander Terrett, Oxfordshire (GB)

Correspondence Address: KLAUBER & JACKSON, 411 HACKENSACK AVENUE, HACKENSACK, NJ, 07601, US

(21) Appl. No.: 10/510,508

(22) PCT Filed: Apr. 11, 2003
(86) PCT No.: PCT/GB03/01589

§ 371 Date: Sep. 12, 2005

(30) Foreign Application Priority Data
Apr. 11, 2002 (GB) 0208332.7
Dec. 21, 2002 (GB) 0229875.0

(51) Int. Cl. G01N 33/574; C12Q 1/68; A61K 39/395; A61K 48/00

(52) U.S. Cl. 424/155.1; 530/388.8; 435/6; 435/7.23; 514/44

(57) ABSTRACT

The present invention relates to new uses of a protein (NKCC1) in the diagnosis, screening, treatment and prophylaxis of breast, lung and/or pancreatic cancer. The invention also provides compositions comprising the protein, including vaccines, antibodies that are immunospecific for the protein and agents which interact with or modulate the expression or activity of the protein or which modulate the expression of the nucleic acid which codes for the protein.

The present invention relates to new uses of a protein (NKCC1) in the diagnosis, screening, treatment and prophylaxis of breast, lung and/or pancreatic cancer. The invention also provides compositions comprising the protein, including vaccines, antibodies that are immunospecific for the protein and agents which interact with or modulate the expression or activity of the protein or which modulate the expression of the nucleic acid which codes for the protein.

The major challenges in breast, lung and/or pancreatic cancer treatment is to improve early detection rates, to find new non-invasive markers that can be used to follow disease progression and identify relapse, and to find improved and less toxic therapies, especially for more advanced disease where 5 year survival is still very poor. There is a great need to identify targets which are more specific to the cancer cells, ideally ones which are expressed on the surface of the tumour cells so that they can be attacked by promising new approaches like immunotherapeutics and targeted toxins.

An ideal protein target for cancer immunotherapy should have a restricted expression profile in normal tissues and be over-expressed in tumours, such that the immune response will be targeted to tumour cells and not against other organs. In addition, the protein target should be exposed on the cell surface, where it will be accessible to therapeutic agents. Tumour specific proteins have been identified for a number of cancer types, by using techniques such as differential screening of cDNA (Hubert, R. S., et al., 1999, Proc. Natl. Acad, Sci. USA 96, 14523-14528; Lucas, S., et al., 2000, Int. J. Cancer 87, 55-60), and the purification of cell-surface proteins that are recognised by tumour-specific antibodies (Catimel, B., et al., 1996, J. Biol. Chem. 271, 25664-25670).

Recently, DNA ‘chips’ containing up to 10,000 expressed sequence elements have been used to characterise tumour cell gene expression (Dhanasekaran, S. M., et al., 2001, Nature 412, 822-826). However, there are several reasons why the numerous and extensive previous transcriptomic analysis of cancers, including breast cancer, may not have revealed all, or even most, tumour associated proteins. These include; (i) a lack of correlation between transcript and disease-associated protein levels, particularly common for membrane proteins that often have a long half-life and as such do not have a high mRNA turnover. Therefore, whilst the difference in protein levels between normal and cancerous cells are consistent it is often difficult to associate changes in the mRNA for a given membrane protein with the cancerous state. (ii) Translocation of a protein in the disease state rather than simply differential levels of the transcript, for example, erbB2/HER2-neu, shows much greater plasma-membrane localisation in cancer cells than normal breast cells, and the transcription factors oestrogen receptor and STAT3 translocate to the nucleus to exert their tumourigenic effects. (iii) Novel/uncharacterised genes are not highly represented within the ‘closed system’ of a cDNA array where there are restrictions on the number of expressed sequence elements per chip and the knowledge and availability of DNA clones.

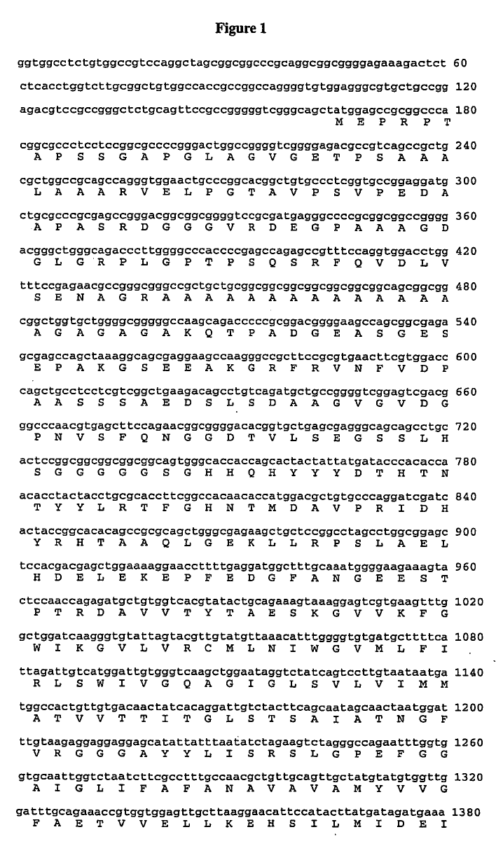

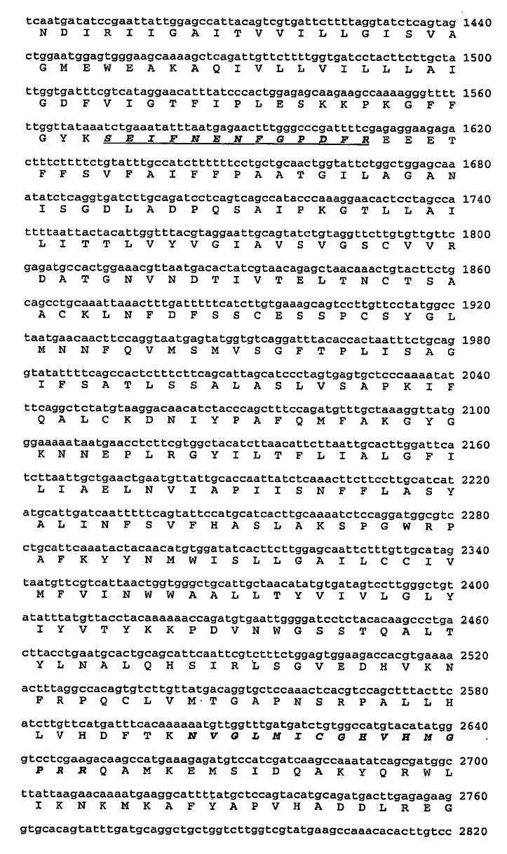

NKCC1 (the sequence of which can be found under the accession number P55011 in the SwissProt database, (available at http://www.expasy.org/), and which is shown in FIG. 1, SEQ ID NO:1) is a bumetanide sensitive sodium-potassium-chloride (Na—K—Cl) cotransporter (Payne et al, 1995, J Biol Chem, 270(30): pp 17977-17985). Two isoforms of the Na—K—Cl cotransporter have been identified; one located on the apical membrane of absorptive epithelia (NKCC2) and one located on the basolateral membrane of secretory epithelia (NKCC1). The function of the Na—K—Cl cotransporters is to provide electroneutral transport of chloride ions across epithelia (in a ratio of 1Na: 1K:2Cl), they work in combination with the sodium and potassium channels and the sodium pump to cause a net transport of sodium chloride across membranes. Dysregulation of this transport mechanism can result in diseases such as Cystic Fibrosis and secretory diarrhoea.

The human NKCC1 protein was identified by the Payne et al in 1995 (see above) by the screening of a human colon carcinoma cell line (T84). It shows significant similarity to the NKCC1 proteins identified from elasmobranch, mouse, rat, rabbit and flounder.

Both isoforms of the Na—K—Cl cotransporter show a similar structure with large N and C terminal domains and 12 transmembrane segments. NKCC1 is significantly larger than NKCC2 with an extra 80 amino acids at the N terminus, interestingly it is the N terminus that shows the greatest variation between species indicating that this section may not be directly involved in the ion transportation. A recent study has indicated that NKCC1 may have 2 splice variants which show differential expression across a variety of tissue types indicating that differential splicing may play a regulatory role in NKCC1 activity (Vibat, et al., Anal Biochem 2001;298(2):218-30).

Over-expression of NKCC1 has been reported in asthmatic subjects by gene expression profiling (Dolganov G M et al, Genome Res (2001); 11(9): 1473-83). This data was then confirmed using immunohistochemistry, which demonstrated that in asthmatic subjects NKCC1 expression is increased with restricted localisation to goblet cells implicating NKCC1 in the pathophysiology of mucus hypersecretion in asthma.

However, no cancer-associated alteration in the expression of the NKCC1 protein indicating the usefulness of NKCC1 in the treatment of breast, lung and/or pancreatic cancer has previously been shown.

The present invention is based on the finding that the expression of NKCC1 protein is increased in breast, lung and/or pancreatic cancer and NKCC1 mRNA shows restricted expression in a few tissues with elevated expression in breast, lung and/or pancreatic cancer, suggesting its utility as a target for breast, lung and/or pancreatic cancer therapy and diagnosis.

Thus, one aspect of the present invention provides a method of screening for and/or diagnosis of breast, lung and/or pancreatic cancer in a subject and/or monitoring the effectiveness of breast, lung and/or pancreatic cancer therapy, which method comprises the step of detecting and/or quantifying in a biological sample obtained from said subject, an NKCC1 polypeptide which:
a) comprises or consists of the amino acid sequence shown in FIG. 1 (SEQ ID NO:1);
b) is a derivative having one or more amino acid substitutions, modifications, deletions or insertions relative to the amino acid sequence shown in FIG. 1 (SEQ ID NO:1) which retains the immunological or biological activity of NKCC1; or
c) is a fragment of a polypeptide having the sequence shown in FIG. 1 (SEQ ID NO: 1), which is at least ten amino acids long and has at least 70% sequence identity over the length of the fragment.

The polypeptides described in a) to c) above are hereinafter referred to as “NKCC1 polypeptides”. The term “polypeptides” includes peptides, polypeptides and proteins. These are used interchangeably unless otherwise specified.

In the context of the present invention, the biological sample can be obtained from any source, such as a serum sample or a tissue sample, e.g. breast, lung, or pancreatic tissue.

A convenient means for detecting/quantifying the NKCC1 polypeptides involves the use of antibodies, therefore, the NKCC1 polypeptides as defined herein also find use in raising antibodies. Thus, in a further aspect, the present invention provides the use of an antibody which binds to at least one NKCC1 polypeptide for screening for and/or diagnosis of breast, lung and/or pancreatic cancer in a subject. Preferably, the antibody is used for detecting and/or quantifying the amount of an NKCC1 polypeptide in a biological sample obtained from said subject.

In one embodiment, binding of antibody in tissue sections can be used to detect aberrant polypeptide localisation or an aberrant level of polypeptide. In a specific embodiment, an antibody to an NKCC1 polypeptide can be used to assay a patient tissue (e.g. a breast, lung and/or pancreatic biopsy) for the level of the polypeptide where an aberrant level of polypeptide is indicative of breast, lung and/or pancreatic cancer. As used herein, an “aberrant level” means a level that is increased or decreased compared with the level in a subject free from breast, lung and/or pancreatic cancer or a reference level. In a specific embodiment an antibody to an NKCC1 polypeptide can be used to assay a patient tissue (e.g. a breast, lung and/or pancreatic biopsy) for the level of the polypeptide where an increased level of the NKCC1 polypeptide is indicative of breast, lung and/or pancreatic cancer.

Suitable immunoassays include, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

In another aspect, the invention provides a method of screening for and/or diagnosis of breast, lung and/or pancreatic cancer in a subject and/or monitoring the effectiveness of breast, lung and/or pancreatic cancer therapy, which method comprises the step of detecting and/or quantifying in a biological sample obtained from said subject, an NKCC1 nucleic acid which:
d) comprises or consists of the DNA sequence shown in FIG. 1 (SEQ ID NO:2) or its RNA equivalent;
e) is a sequence which codes for a polypeptide as defined in a), b) or c);
f) is a sequence which is complementary to the sequences of d) or e);
g) is a sequence which codes for the same polypeptide, as the sequences of d) or e);
h) is a sequence which shows substantial identity with any of those of d), e), f) or g); or
i) is a fragment of d), e), f), g) or h), which is at least 8 nucleotides in length.

The term ‘RNA equivalent’ when used above indicates that a given RNA molecule has a sequence which is complementary to that of a given DNA molecule, allowing for the fact that in RNA ‘U’ replaces ‘T’ in the genetic code. The nucleic acid molecule may be in isolated, recombinant or chemically synthetic form.

Unless the context indicates otherwise, the term “NKCC1 nucleic acid” includes those nucleic acid molecules defined in d) to i) above and may have one or more of the following characteristics:
1) they may be DNA or RNA;
2) they may be single or double stranded;
3) they may be provided in recombinant form, e.g. covalently linked to a 5′ and/or a 3′ flanking sequence to provide a molecule which does not occur in nature;
4) they may be provided without 5′ and/or 3′ flanking sequences which normally occur in nature;
5) they may be provided in substantially pure form. Thus they may be provided in a form which is substantially free from contaminating proteins and/or from other nucleic acids; and
6) they may be provided with introns or without introns (e.g. as cDNA).

In view of the foregoing description the skilled person will appreciate that utilisation of a large number of nucleic acids is within the scope of the present invention.

In another aspect, the present invention provides a method for the prophylaxis and/or treatment of breast, lung and/or pancreatic cancer in a subject, which comprises administering to said subject a therapeutically effective amount of an NKCC1 polypeptide or an NKCC1 nucleic acid.

In a yet another aspect, the present invention provides the use of an NKCC1 polypeptide or an NKCC1 nucleic acid in the preparation of a composition for use in the prophylaxis and/or treatment of breast, lung and/or pancreatic cancer. The subject may be a mammal and is preferably a human.

In the aspects supra, the NKCC1 polypeptides may be provided in isolated or recombinant form, and may be fused to other moieties. The NKCC1 polypeptides thereof may be provided in substantially pure form, that is to say free, to a substantial extent, from other proteins. Thus, an NKCC1 polypeptide may be provided in a composition in which it is the predominant component present (i.e. it is present at a level of at least 50%; preferably at least 75%, at least 80%, at least 85%, at least 90%, at least 95% or at least 98%; when determined on a weight/weight basis excluding solvents or carriers).

In order to more fully appreciate the present invention, polypeptides within the scope of a)-c) above will now be discussed in greater detail.

Polypeptides within the Scope of a)

A polypeptide within the scope of a), may consist of the particular amino acid sequence given in FIG. 1 (SEQ ID NO: 1) or may have an additional N-terminal and/or an additional C-terminal amino acid sequence relative to the sequence given in FIG. 1 (SEQ ID NO: 1). Additionally, NKCC1 polypeptides may be in the form of a “mature” protein or may be part of a larger protein such as a fusion protein.

Additional N-terminal or C-terminal sequences may be provided for various reasons, it is often advantageous to include an additional amino acid sequence which contains secretory or leader sequences, a pre-, pro- or prepro-protein sequence, or a sequence which aids in purification, such as an affinity tag. An additional sequence which may provide stability during recombinant production may also be used. Such sequences may be optionally removed as required by incorporating a cleavable sequence as an additional sequence or part thereof. Thus an NKCC1 polypeptide may be fused to other moieties including other polypeptides. Such additional sequences and affinity tags are well known in the art. Techniques for providing such additional sequences are well known in the art.

Additional sequences may be provided in order to alter the characteristics of a particular polypeptide. This can be useful in improving expression or regulation of expression in particular expression systems. For example, an additional sequence may provide some protection against proteolytic cleavage.

Additional sequences can also be useful in altering the properties of a polypeptide to aid in identification or purification. For example, a fusion protein may be provided in which a polypeptide is linked to a moiety capable of being isolated by affinity chromatography, for example, but without limitation, multiple histidine residues, a FLAG tag, HA tag or myc tag. The moiety may be an antigen or an epitope and the affinity column may comprise immobilised antibodies or immobilised antibody fragments which bind to said antigen or epitope (desirably with a high degree of specificity). The fusion protein can usually be eluted from the column by addition of an appropriate buffer.

Additional N-terminal or C-terminal sequences may, however, be present simply as a result of a particular technique used to obtain a polypeptide and need not provide any particular advantageous characteristic to the polypeptide. Such polypeptides are within the scope of the present invention.

Whatever additional N-terminal or C-terminal sequence is present, it is preferred that the resultant polypeptide should exhibit the immunological or biological activity of the polypeptide having the amino acid sequence shown in FIG. 1 (SEQ ID NO: 1).

Polypeptides within the Scope of b)

Turning now to the polypeptides defined in b) above, it will be appreciated by the person skilled in the art that these polypeptides are derivatives of the polypeptide given in a) above, provided that such derivatives preferably exhibit the immunological or biological activity of the polypeptide having the amino acid sequence shown in FIG. 1 (SEQ ID NO: 1). Alternatively, the biological activity of the polypeptide may be altered. As such, it will be appreciated by one skilled in the art that derivatives can include post-translational modifications, for example but without limitation, phosphorylation, glycosylation and farnesylation. Modifications include naturally occurring modifications, such as, without limitation, post-translational modifications, and non-naturally occurring modifications such as may be introduced by mutagenesis.

Alterations in the amino acid sequence of a protein can occur which do not affect the activity of a protein. These include amino acid deletions, insertions and substitutions and can result from alternative splicing and/or the presence of multiple translation start sites and stop sites. Polymorphisms may arise as a result of the infidelity of the translation process. Thus changes in amino acid sequence may be tolerated which do not affect the protein's biological or immunological activity.

In further aspects of the present invention, for example therapy and/or prophylaxis of breast, lung and/or pancreatic cancer the use of dominant negative or constitutively active NKCC1 polypeptides is contemplated. Therefore, in one embodiment, the deleted, inserted, modified or substituted amino acid(s) renders dominant negative activity upon the peptide. In another embodiment, the deleted, inserted, modified or substituted amino acid(s) renders the polypeptide constitutively active.

The skilled person will appreciate that various changes can often be made to the amino acid sequence of a polypeptide which has a particular activity to produce derivatives (sometimes known as variants or “muteins”) having at least a proportion of said activity, and preferably having a substantial proportion of said activity. Such derivatives of the polypeptides described in a) above are within the scope of the present invention and are discussed in greater detail below. They include allelic and non-allelic derivatives.

An example of a derivative of an NKCC1 polypeptide is a polypeptide as defined in a) above, apart from the substitution of one or more amino acids with one or more other amino acids. The skilled person is aware that various amino acids have similar properties. One or more such amino acids of a polypeptide can often be substituted by one or more other such amino acids without eliminating a desired activity of that polypeptide.

Thus, the amino acids glycine, alanine, valine, leucine and isoleucine can often be substituted for one another (amino acids having aliphatic side chains). Of these possible substitutions, it is preferred that glycine and alanine are used to substitute for one another (since they have relatively short side chains) and that valine, leucine and isoleucine are used to substitute for one another (since they have larger aliphatic side chains which are hydrophobic).

Other amino acids which can often be substituted for one another include:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains);
cysteine and methionine (amino acids having sulphur-containing side chains); and
aspartic acid and glutamic acid can substitute for phospho-serine and phospho-threonine, respectively (amino acids with acidic side chains).

Substitutions of this nature are often referred to as “conservative” or “semi-conservative” amino acid substitutions.

Amino acid deletions or insertions may also be made relative to the amino acid sequence given in a) above. Thus, for example, amino acids which do not have a substantial effect on the biological and/or immunological activity of the polypeptide, or at least which do not eliminate such activity, may be deleted. Such deletions can be advantageous since the overall length and the molecular weight of a polypeptide can be reduced whilst still retaining activity. This can enable the amount of polypeptide required for a particular purpose to be reduced for example, dosage levels can be reduced.

Amino acid insertions relative to the sequence given in a) above can also be made. This may be done to alter the properties of an NKCC1 polypeptide (e.g. to assist in identification, purification or expression, as explained above in relation to fusion proteins).

Amino acid changes relative to the sequence given in a) above can be made using any suitable technique, e.g. by using site-directed mutagenesis (Hutchinson et al., 1978, J. Biol. Chem. 253:6551).

It should be appreciated that amino acid substitutions or insertions to the polypeptide for use in the present invention can be made using naturally occurring or non-naturally occurring amino acids. Whether or not natural or synthetic amino acids are used, it is preferred that only L-amino acids are present.

Whatever amino acid changes are made (whether by means of substitution, modification, insertion or deletion), preferred NKCC1 polypeptides have at least 50% sequence identity with a polypeptide as defined in a) above, more preferably the degree of sequence identity is at least 75%, at least 80%, at least 85%. Sequence identities of at least 90%, at least 95% or at least 98% are most preferred.

The term identity can be used to describe the similarity between two polypeptide sequences. The degree of amino acid sequence identity can be calculated using a program such as “bestfit” (Smith and Waterman, Advances in Applied Mathematics, 482-489 (1981)) to find the best segment of similarity between any two sequences. The alignment is based on maximising the score achieved using a matrix of amino acid similarities, such as that described by Schwarz and Dayhof (1979) Atlas of Protein Sequence and Structure, Dayhof, M. O., Ed pp 353-358.

A software package well known in the art for carrying out this procedure is the CLUSTAL program. It compares the amino acid sequences of two polypeptides and finds the optimal alignment by inserting spaces in either sequence as appropriate. The amino acid identity or similarity (identity plus conservation of amino acid type) for an optimal alignment can also be calculated using a software package such as BLASTX. This program aligns the largest stretch of similar sequence and assigns a value to the fit. For any one pattern comparison, several regions of similarity may be found, each having a different score. One skilled in the art will appreciate that two polypeptides of different lengths may be compared over the entire length of the longer fragment. Alternatively small regions may be compared. Normally sequences of the same length are compared for a useful comparison to be made.

Where high degrees of sequence identity are present there will be relatively few differences in amino acid sequence. Thus for example they may be less than 20, less than 10, or even less than 5 differences.

Polypeptides within the Scope of c)

As discussed supra, it is often advantageous to reduce the length of a polypeptide, preferably the resultant reduced length polypeptide still has a desired activity or can give rise to useful antibodies. Feature c) therefore covers fragments of polypeptides a) or b) above for use in the present invention.

As used herein, the term “fragment” refers to a polypeptide comprising an amino acid sequence of at least 10 amino acid residues (preferably, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, at least 250 amino acid residues, at least 300 amino acid residues, at least 350 amino acid residues, at least 400 amino acid residues, at least 450 amino acid residues, at least 500 amino acid residues, at least 550 amino acid residues, at least 600 amino acid residues, at least 650 amino acid residues, at least 700 amino acid residues, at least 750 amino acid residues, at least 800 amino acid residues, at least 850 amino acid residues, at least 900 amino acid residues, at least 950 amino acid residues, at least 1000 amino acid residues, at least 1050 amino acid residues, at least 1100 amino acid residues, at least 1150 amino acid residues or at least 1200 amino acid residues) of the amino acid sequence of a parent polypeptide. Any given fragment of the NKCC1 polypeptide may or may not possess the functional activity of the parent polypeptide. A fragment has at least 70% identity over its length to the amino acid sequence shown in FIG. 1 (SEQ ID NO: 1), preferably it has at least 75%, at least 80%, at least 85%, at least 90%, at least 95% or at least 98% identity. A skilled person can determine whether or not a particular fragment has activity.

An NKCC1 polypeptide may be useful as antigenic material, and may be used in the production of vaccines for treatment or prophylaxis of breast, lung and/or pancreatic cancer. Such material can be “antigenic” and/or “immunogenic”. Generally, “antigenic” is taken to mean that the protein is capable of being used to raise antibodies or indeed is capable of inducing an antibody response in a subject. “Immunogenic” is taken to mean that the protein is capable of eliciting an immune response in a subject. Thus, in the latter case, the protein may be capable of not only generating an antibody response but, in addition, non-antibody based immune responses.

It is well known that it is possible to screen an antigenic protein or polypeptide to identify epitopic regions, i.e. those regions which are responsible for the protein or polypeptide's antigenicity or immunogenicity. Methods well known to the skilled person can be used to test fragments and/or homologues and/or derivatives for antigenicity. Thus, the fragments of NKCC1 polypeptides may include one or more such epitopic regions or be sufficiently similar to such regions to retain their antigenic/immunogenic properties. Thus, for fragments of NKCC1 polypeptides the degree of identity is perhaps irrelevant, since they may be 100% identical to a particular part of a protein or polypeptide, homologue or derivative as described herein. The key issue may be that the fragment retains the antigenic and/or immunogenic properties of the protein from which it is derived.

Homologues, derivatives and fragments may possess at least a degree of the antigenicity and/or immunogenicity of the protein from which they are derived.

As will be discussed below, NKCC1 polypeptides find use in a therapeutic approach to breast, lung and/or pancreatic cancer. In a particular embodiment, an NKCC1 polypeptide is fused to another polypeptide, such as the protein transduction domain of the HIV/Tat protein, which facilitates the entry of fusion protein into a cell (Asob, S. et al., 2002, Proc. Natl. Acad. Sci. USA, 99:17107-17112). Such a fusion protein may be provided for use in the preparation of a composition for the treatment of breast, lung and/or pancreatic cancer. The skilled person will appreciate that for the preparation of one or more such polypeptides, the preferred approach will be based on recombinant DNA techniques.

The NKCC1 polypeptides can be coded for by a large variety of nucleic acid molecules, taking into account the well-known degeneracy of the genetic code. All of these molecules can be used in the present invention. They can be inserted into vectors and cloned to provide large amounts of DNA or RNA for further study. Suitable vectors may be introduced into host cells to enable the expression of polypeptides used in the present invention using techniques known to the person skilled in the art.

Techniques for cloning, expressing and purifying proteins and polypeptides are well known to the skilled person. DNA constructs can readily be generated using methods well known in the art. These techniques are disclosed, for example in J. Sambrook et al, Molecular Cloning 2^{nd} Edition, Cold Spring Harbour Laboratory Press (1989); in Old & Primrose Principles of Gene Manipulation 5th Edition, Blackwell Scientific Publications (1994); and in Stryer Biochemistry 4th Edition, W H Freeman and Company (1995). Modifications of DNA constructs and the proteins expressed such as the addition of promoters, enhancers, signal sequences, leader sequences, translation start and stop signals and DNA stability controlling regions, or the addition of fusion partners may then be facilitated.

Normally the DNA construct will be inserted into a vector, which may be of phage or plasmid origin. Expression of the protein is achieved by the transformation or transfection of the vector into a host cell, which may be of eukaryotic or prokaryotic origin. Such vectors and suitable host cells form further aspects for use in the present invention.

For recombinant NKCC1 polypeptide production, host cells can be genetically engineered to incorporate expression systems or portions thereof for NKCC1 nucleic acids. Such incorporation can be performed using methods well known in the art, such as, calcium phosphate transfection, DEAE-dextran mediated transfection, transvection, microinjection, cationic lipid mediated transfection, electroporation, transduction, scrape loading, ballistic introduction or infection (see e.g. Davis et al., Basic Methods in Molecular Biology, 1986 and Sambrook et al., Molecular Cloning: a Laboratory Manual, 2^{nd} Ed., Cold Spring Harbor Laboratory Press, Cold Spring Harbor, N.Y., 1989).

Representative examples of host cells include bacterial cells e.g. E. Coli, Streptococci, Staphylococci, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, HEK 293, BHK and Bowes melanoma cells; and plant cells.

A wide variety of expression systems can be used, such as and without limitation, chromosomal, episomal and virus-derived systems, e.g. vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a nucleic acid to produce a polypeptide in a host may be used. The appropriate nucleic acid sequence may be inserted into an expression system by any variety of well-known and routine techniques, such as those set forth in Sambrook et al., supra. Appropriate secretion signals may be incorporated into the NKCC1 polypeptide to allow secretion of the translated protein into the lumen of the endoplasmic reticulum, the periplasmic space or the extracellular environment. These signals may be endogenous to the NKCC1 polypeptide or they may be heterologous signals.

Cell-free translation systems can also be employed to produce recombinant polypeptides (e.g. rabbit reticulocyte lysate, wheat germ lysate, SP6/T7 in vitro T & T and RTS 100 E. Coli HY coupled Transcription/Translation kits from Roche Diagnostics Ltd., Lewes, UK and the TNT Quick coupled Transcription/Translation System from Promega UK, Southampton, UK.

The NKCC1 nucleic acids may be synthesised using methods known in the art, such as using conventional chemical approaches or polymerase chain reaction (PCR) amplification. The NKCC1 nucleic acids also permit the identification and cloning of the gene encoding an NKCC1 polypeptide from any species, for instance by screening cDNA libraries, genomic libraries or expression libraries.

Knowledge of the nucleic acid structure can be used to raise antibodies and for gene therapy. Techniques for this are well-known by those skilled in the art, as discussed in more detail herein.

By using appropriate expression systems, NKCC1 polypeptides may be expressed in glycosylated or non-glycosylated form. Non-glycosylated forms can be produced by expression in prokaryotic hosts, such as E. coli.

Polypeptides comprising N-terminal methionine may be produced using certain expression systems, whilst in others the mature polypeptide will lack this residue.

Preferred techniques for cloning, expressing and purifying an NKCC1 polypeptide are summarised below:

Polypeptides may be prepared natively or under denaturing conditions and then subsequently refolded. Baculoviral expression vectors include secretory plasmids (such as pACGP67 from Pharmingen), which may have an epitope tag sequence cloned in frame (e.g. myc, V5 or His) to aid detection and allow for subsequent purification of the protein. Mammalian expression vectors may include pCDNA3 and pSecTag (both Invitrogen), and pREP9 and pCEP4 (Invitrogen). E. coli systems include the pBad series (His tagged-Invitrogen) or pGex series (Pharmacia).

In one embodiment, NKCC1 polypeptides are provided in isolated form and include NKCC1 polypeptides that have been purified to at least some extent. NKCC1 polypeptides can be produced using recombinant methods, synthetically produced or produced by a combination of these methods. NKCC1 polypeptides may be provided in substantially pure form, that is to say free, to a substantial extent, from other proteins.

If an NKCC1 polypeptide is to be expressed for use in cell-based screening assays, as described below, it is preferred that the polypeptide is produced at the cell surface. In this event, the cells may be harvested prior to use in the screening assay. If the NKCC1 polypeptide is secreted into the medium, the medium can be recovered in order to isolate said polypeptide. If produced intracellularly, the cells must first be lysed before the NKCC1 polypeptide is recovered.

NKCC1 polypeptides can be recovered and purified from recombinant cell cultures by well-known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to an NKCC1 polypeptide can be used to deplete a sample, comprising an NKCC1 polypeptide, of said polypeptide or to purify said polypeptide. Techniques well-known in the art, may be used for refolding to regenerate native or active conformations of the NKCC1 polypeptides when the polypeptides have been denatured during isolation and or purification.

In addition to nucleic acid molecules coding for NKCC1 polypeptides, referred to herein as “coding” nucleic acids, the present invention also utilises nucleic acids complementary thereto. Thus, for example, both strands of a double stranded nucleic acid molecule are included in the present invention (whether or not they are associated with one another). Also included are mRNA molecules and complementary DNA molecules (e.g. cDNA molecules).

The use of nucleic acid molecules which can hybridise to any of the nucleic acid molecules discussed above, in the diagnosis, screening, treatment and/or prophylaxis of breast, lung and/or pancreatic cancer is also covered by the present invention. Such nucleic acid molecules are referred to herein as “hybridising” nucleic acid molecules. Hybridising nucleic acid molecules can be useful as probes or primers, for example.

Desirably such hybridising molecules are at least 8 nucleotides in length and preferably are at least 25 or at least 50 nucleotides in length. The hybridising nucleic acid molecules preferably hybridise to nucleic acids within the scope of d), e), f), g), h) or i) above specifically.

Desirably the hybridising molecules will hybridise to such molecules under stringent hybridisation conditions. One example of stringent hybridisation conditions is where attempted hybridisation is carried out at a temperature of from about 35° C. to about 65° C. using a salt solution that is about 0.9 molar. However, the skilled person will be able to vary such conditions as appropriate in order to take into account variables such as probe length, base composition, type of ions present, etc. For a high degree of selectivity, relatively stringent conditions are used to form the duplexes, such as low salt or high temperature conditions. As used herein, “highly stringent conditions” means hybridisation to filter-bound DNA in 0.5 M NaHPO₄, 7% sodium dodecyl sulphate (SDS), 1 mM EDTA at 65° C., and washing in 0.1×SSC/0.1% SDS at 68° C. (Ausubel P. M. et al., eds., 1989, Current Protocols in Molecular Biology, Vol. I, Green Publishing Associates, Inc., and John Wiley & Sons, Inc., New York, at p. 2.10.3). For some applications, less stringent conditions for duplex formation are required. As used herein “moderately stringent conditions” means washing in 0.2×SSC/0.1% SDS at 42° C. (Ausubel et al., 1989, supra). Hybridisation conditions can also be rendered more stringent by the addition of increasing amounts of formamide, to destabilise the hybrid duplex. Thus, particular hybridisation conditions can be readily manipulated, and will generally be chosen depending on the desired results. In general, convenient hybridisation temperatures in the presence of 50% formamide are: 42° C. for a probe which is 95 to 100% identical to the fragment of a gene encoding an NKCC1 polypeptide, 37° C. for 90 to 95% identity and 32° C. for 70 to 90% identity. In the preparation of genomic libraries, DNA fragments are generated, some of which will encode parts or the whole of an NKCC1 polypeptide. The DNA may be cleaved at specific sites using various restriction enzymes. Alternatively, one may use DNAse in the presence of manganese to fragment the DNA, or the DNA can be physically sheared, for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T₄, and yeast artificial chromosomes (YACs). (See, for example, Sambrook et al., 1989, Molecular Cloning, A Laboratory Manual, 1D Ed., Cold Spring Harbor Laboratory Press, Cold Spring Harbor, N.Y.; Glover, D. M. (ed.), 1985, DNA Cloning: A Practical Approach, MRL Press, Ltd, Oxford, U.K. Vol. I, II; Ausubel F. M. et al., eds., 1989, Current Protocols in Molecular Biology, Vol. I, Green Publishing Associates, Inc., and John Wiley & sons, Inc., New York). The genomic library may be screened by nucleic acid hybridisation to labelled probe (Benton & Davis, 1977, Science 196:180; Grunstein & Hogness, 1975, Proc. Natl. Acad. Sci. U.S.A. 72:3961).

Manipulation of the DNA encoding a protein is a particularly powerful technique for both modifying proteins and for generating large quantities of protein for purification purposes. This may involve the use of PCR techniques to amplify a desired nucleic acid sequence. Thus the sequence data provided herein can be used to design primers for use in PCR so that a desired sequence can be targeted and then amplified to a high degree.

Typically, primers will be at least eight nucleotides long and will generally be at least ten nucleotides long (e.g. fifteen to twenty-five nucleotides long). In some cases, primers of at least thirty or at least thirty-five nucleotides in length may be used.

As a further alternative chemical synthesis may be used, this may be automated. Relatively short sequences may be chemically synthesised and ligated together to provide a longer sequence.

The term identity can also be used to describe the similarity between two individual DNA sequences. The ‘bestfit’ program (Smith and Waterman, 1981, Advances in applied Mathematics, 482-489) is one example of a type of computer software used to find the best segment of similarity between two nucleic acid sequences, whilst the GAP program enables sequences to be aligned along their whole length and finds the optimal alignment by inserting spaces in either sequence as appropriate. It is preferred if sequences which show substantial identity with any of those of d), e) and f) have e.g. at least 50%, at least 75%, at least 80%, at least 85%, at least 90%, at least 95% or at least 98% sequence identity.

A hybridising nucleic acid molecule of use in the present invention may have a high degree of sequence identity along its length with a nucleic acid molecule within the scope of d)-i) above (e.g. at least 50%, at least 75%, at least 80%, at least 85%, at least 90%, at least 95% or at least 98% sequence identity). As will be appreciated by the skilled person, the higher the sequence identity a given single stranded nucleic acid molecule has with another nucleic acid molecule, the greater the likelihood that it will hybridise to a nucleic acid molecule which is complementary to that other nucleic acid molecule under appropriate conditions.

If desired, a gene encoding an NKCC1 polypeptide, a related gene, or related nucleic acid sequences or subsequences, including complementary sequences, can also be, used in hybridisation assays. A nucleotide encoding an NKCC1 polypeptide, or subsequences thereof comprising at least 8 nucleotides, can be used as a hybridisation probe. Hybridisation assays can be used for detection, prognosis, diagnosis, or monitoring of conditions, disorders, or disease states, associated with aberrant expression of genes encoding an NKCC1 polypeptide, or for differential diagnosis of patients with signs or symptoms suggestive of breast, lung and/or pancreatic cancer.

In particular embodiment, such a hybridisation assay can be carried out by a method comprising:
i) contacting a biological sample containing nucleic acid with a nucleic acid probe capable of hybridising to a DNA or RNA that encodes an NKCC1 polypeptide, under conditions such that hybridisation can occur; and
ii) detecting or measuring any resulting hybridisation.

The invention also provides a kit comprising a nucleic acid probe capable of hybridising to RNA encoding an NKCC1 polypeptide. In a specific embodiment, a kit comprises in one or more containers a pair of primers (e.g. each in the size range of 6-30 nucleotides, more preferably 10-30 nucleotides and still more preferably 10-20 nucleotides) that under appropriate reaction conditions can prime amplification of at least a portion of a nucleic acid encoding an NKCC1 polypeptide, such as by polymerase chain reaction (see e.g. Innis et al., 1990, PCR Protocols, Academic Press, Inc., San Diego, Calif.), ligase chain reaction (see EP 320308) use of Qβ replicase, cyclic probe reaction, or other methods known in the art.

In another embodiment, a preparation of oligonucleotides comprising 10 or more consecutive nucleotides complementary to a nucleotide sequence encoding an NKCC1 polypeptide for use as vaccines for the treatment of breast, lung and/or pancreatic cancer. Such preparations may include adjuvants or other vehicles.

In yet another embodiment, the present invention provides the use of at least one NKCC1 nucleic acid in the preparation of a pharmaceutical composition for use in the treatment of breast, lung and/or pancreatic cancer.

In addition to being used as primers and/or probes, hybridising nucleic acid molecules can be used as anti-sense molecules to alter the expression of NKCC1 polypeptides by binding to complementary nucleic acid molecules. This technique can be used in antisense therapy.

As used herein, an “anti-sense” nucleic acid refers to a nucleic acid capable of hybridising by virtue of some sequence complementarity to a portion of an RNA (preferably mRNA) encoding a polypeptide as defined herein. The antisense nucleic acid may be complementary to a coding and/or non-coding region of a mRNA encoding such a polypeptide. Such antisense nucleic acids have utility as compounds that inhibit expression, and can be used in the treatment or prevention of breast, lung and/or pancreatic cancer.

In a specific embodiment, expression of an NKCC1 polypeptide is inhibited by use of antisense nucleic acids. The present invention provides the therapeutic or prophylactic use of nucleic acids comprising at least eight nucleotides that are antisense to a gene or cDNA encoding an NKCC1 polypeptide.

Endogenous polypeptide expression can also be reduced by inactivating or “knocking out” the gene encoding the polypeptide, or the promoter of such a gene, using targeted homologous recombination (e.g. see Smithies, et al., 1985, Nature 317:230-234; Thomas & Capecchi, 1987, Cell 51:503-512; Thompson et al., 1989, Cell 5:313-321; and Zijlstra et al., 1989, Nature 342:435-438). For example, a mutant gene encoding a non-functional polypeptide (or a completely unrelated DNA sequence) flanked by DNA homologous to the endogenous gene (either the coding regions or regulatory regions of the gene encoding the polypeptide) can be used, with or without a selectable marker and/or a negative selectable marker, to transfect cells that express the target gene in vivo. Insertion of the DNA construct, via targeted homologous recombination, results in inactivation of the target gene. This approach can be adapted for use in humans, provided the recombinant DNA constructs are directly administered or targeted to the required site in vivo using appropriate viral vectors.

In another embodiment, symptoms of breast, pancreatic and/or lung cancer may be ameliorated by decreasing the level or activity of an NKCC1 polypeptide by using gene sequences encoding an NKCC1 polypeptide in conjunction with well-known gene “knock-out”, ribozyme or triple helix methods to decrease gene expression of the polypeptide. In this approach, ribozyme or triple helix molecules are used to modulate the activity, expression or synthesis of the gene, and thus to ameliorate the symptoms of breast, pancreatic and/or lung cancer. Such molecules may be designed to reduce or inhibit expression of a mutant or non-mutant target gene. Techniques for the production and use of such molecules are well known to those of skill in the art.

In a further embodiment, the NKCC1 nucleic acid is administered via gene therapy (see for example Hoshida, T. et al., 2002, Pancreas, 25:111-121; Ikuno, Y. 2002, Invest. Ophthalmol. Vis. Sci. 2002 43:2406-2411; Bollard, C., 2002, Blood 99:3179-3187; Lee E., 2001, Mol. Med. 7:773-782).

Gene therapy refers to administration to a subject of an expressed or expressible nucleic acid. In this embodiment, the nucleic acid produces its encoded protein that mediates a therapeutic effect by promoting polypeptide function. Any of the methods for gene therapy available in the art can be used according to the present invention.

In a preferred aspect, the pharmaceutical composition comprises an NKCC1 nucleic acid, such as a nucleic acid encoding an NKCC1 polypeptide, said nucleic acid being part of an expression vector that expresses an NKCC1 polypeptide or chimeric protein thereof in a suitable host. In particular, such a nucleic acid has a promoter operably linked to the polypeptide coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller & Smithies, 1989, Proc. Natl. Acad. Sci USA 86:8932-8935; Zijlstra et al., 1989, Nature 342:435-438).

Delivery of the nucleic acid into a patient may be direct, in which case the patient is directly exposed to the nucleic acid or nucleic acid-carrying vector; this approach is known as in vivo gene therapy. Alternatively, delivery of the nucleic acid into the patient may be indirect, in which case cells are first transformed with the nucleic acid in vitro and then transplanted into the patient; this approach is known as ex vivo gene therapy.

The present invention also demonstrates that NKCC1 is a suitable immunotherapeutic target for the treatment and/or prophylaxis of breast, pancreatic and/or lung cancer. Therefore, in a further aspect the present invention provides antibodies that recognise NKCC1 polypeptides and their use in the treatment and/or prophylaxis of breast, pancreatic and/or lung cancer.

NKCC1 polypeptides, may be used as immunogens to generate antibodies which immunospecifically bind such an immunogen, these are referred to herein as NKCC1 antibodies. NKCC1 antibodies include, but are not limited to polyclonal, monoclonal, bispecific, humanised or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically-active portions of immunoglobulin molecules, i.e. molecules that contain an antigen binding site that specifically binds an antigen. The immunoglobulin molecules can be of any class (e.g. IgG, IgE, IgM, IgD and IgA) or subclass of immunoglobulin molecule. Preferred antibodies bind specifically to NKCC1 polypeptides so that they can be used to purify and/or inhibit the activity of such polypeptides. Specifically recognising or binding specifically means that the antibodies have a greater affinity for NKCC1 polypeptides than for other polypeptides.

In the production of antibodies, screening for the desired antibody can be accomplished by techniques known in the art, e.g. ELISA (enzyme-linked immunosorbent assay). For example, to select antibodies, which recognise a specific domain of an NKCC1 polypeptide, one may assay generated hybridomas for a product which binds to a polypeptide fragment containing such domain. For selection of an antibody that specifically binds a first polypeptide homologue but which does not specifically bind to (or binds less avidly to) a second polypeptide homologue, one can select on the basis of positive binding to the first polypeptide homologue and a lack of binding to (or reduced binding to) the second polypeptide homologue.

For preparation of NKCC1 monoclonal antibodies (mAbs), any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs for use in the invention may be cultivated in vitro or in vivo. In an additional embodiment of the invention, mAbs can be produced in germ-free animals utilising known technology.

The mAbs include but are not limited to human mAbs and chimeric mAbs (e.g. human-mouse chimeras). A chimeric antibody is a molecule in which different portions are derived from different animal species, such as those having a human immunoglobulin constant region and a variable region derived from a murine mAb, (see, e.g. U.S. Pat. No. 4,816,567; and U.S. Pat. No. 4,816,397). Humanised antibodies are antibody molecules from non-human species having one or more complementarity determining regions (CDRs) from the non-human species and a framework region from a human immunoglobulin molecule, (see, e.g. U.S. Pat. No. 5,585,089).

Chimeric and humanised mAbs can be produced by recombinant DNA techniques known in the art, for example using methods described in WO 87/02671; EP 184187; EP 171496; EP 173494; WO 86/01533; U.S. Pat. No. 4,816,567; EP 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunised in the normal fashion with a selected antigen, e.g. all or a portion of a polypeptide for use in the invention. mAbs directed against the antigen can be obtained using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM, IgD and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g. U.S. Pat. No. 5,625,126; U.S. Pat. No. 5,633,425; U.S. Pat. No. 5,569,825; U.S. Pat. No. 5,661,016; and U.S. Pat. No. 5,545,806.

Completely human antibodies, which recognise a selected epitope, can be generated using a technique referred to as “guided selection”. In this approach a selected non-human monoclonal antibody, e.g. a mouse antibody, is used to guide the selection of a completely human antibody recognising the same epitope, (Jespers et al. (1994) Bio/technology 12:899-903).

The NKCC1 antibodies can also be generated using various phage display methods known in the art. In phage display methods, functional antibody domains are displayed on the surface of phage particles which carry the polynucleotide sequences encoding them. In a particular, such phage can be utilised to display antigen binding domains expressed from a repertoire or combinatorial antibody library (e.g. human or murine). Phage expressing an antigen binding domain that binds the antigen of interest can be selected or identified with antigen, e.g. using labelled antigen or antigen bound or captured to a solid surface or bead. Phage used in these methods are typically filamentous phage including fd and M13 binding domains expressed from phage with Fab, Fv or disulphide stabilised Fv antibody domains recombinantly fused to either the phage gene III or gene VIII protein. Phage display methods that can be used to make the antibodies of the present invention include those disclosed in Brinkman et al., J. Immunol. Methods 182: 41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g. as described in detail below. For example, techniques to recombinantly produce Fab, Fab′ and F(ab′)2 fragments can also be employed using methods known in the art such as those disclosed in WO 92/22324; Mullinax et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. No. 4,946,778, U.S. Pat. No. 5,258,498; Huston et al., Methods in Enzymology 203:46-88 (1991); Shu et al., PNAS 90:7995-7999 (1993); and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details for generating bispecific antibodies see, for example, Suresh et al., Methods in Enzymology, 1986, 121:210.

The invention provides functionally-active fragments, derivatives or analogues of the anti-NKCC1 antibodies. “Functionally-active” means that the fragment, derivative or analogue is able to elicit anti-anti-idiotype antibodies (i.e. tertiary antibodies) that recognise the same antigen that is recognised by the antibody from which the fragment, derivative or analogue is derived. Specifically, in a preferred embodiment, the antigenicity of the idiotype of the immunoglobulin molecule may be enhanced by deletion of framework and CDR sequences that are C-terminal to the CDR sequence that specifically recognises the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)2 fragments and Fab fragments. Antibody fragments which recognise specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulphide bridges of the F(ab′)2 fragments. The invention also provides heavy chain and light chain dimers of the NKCC1 antibodies, or any minimal fragment thereof such as Fvs or single chain antibodies (SCAs) (e.g. as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the NKCC1 antibody. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the NKCC1 antibodies (or functionally active fragments thereof), for example in which the antibody is fused via a covalent bond (e.g. a peptide bond), at either the N-terminus or the C-terminus to an amino acid sequence of another protein (or portion thereof, preferably at least a 10, 20 or 50 amino acid portion of the protein) that is not the antibody. Preferably the antibody, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The NKCC1 antibodies include analogues and derivatives that are modified, e.g. by the covalent attachment of any type of molecule, as long as such covalent attachment that does not impair immunospecific binding. For example, but not by way of limitation, the derivatives and analogues of the antibodies include those that have been further modified, e.g. by glycosylation, acetylation, pegylation, phosphylation, amidation, derivatisation by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analogue or derivative may contain one or more non-classical amino acids.

The foregoing antibodies can be used in methods known in the art relating to the localisation and activity of the NKCC1 polypeptides, e.g. for imaging or radioimaging these proteins, measuring levels thereof in appropriate physiological samples, in diagnostic methods, etc. and for radiotherapy.

The NKCC1 antibodies can be produced by any method known in the art for the synthesis of antibodies, in particular, by chemical synthesis or by recombinant expression, and are preferably produced by recombinant expression technique.

Recombinant expression of antibodies, or fragments, derivatives or analogues thereof, requires construction of a nucleic acid that encodes the antibody. If the nucleotide sequence of the antibody is known, a nucleic acid encoding the antibody may be assembled from chemically synthesised oligonucleotides (e.g. as described in Kutmeier et al., 1994, BioTechniques 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g. an antibody cDNA library, or cDNA library generated from any tissue or cells expressing the antibody) by PCR amplification using synthetic primers hybridisable to the 3′ and 5′ ends of the sequence or by cloning using an oligonucleotide probe specific for the particular gene sequence.

If an antibody molecule that specifically recognises a particular antigen is not available (or a source for a cDNA library for cloning a nucleic acid encoding such an antibody), antibodies specific for a particular antigen may be generated by any method known in the art, for example, by immunising an animal, such as a rabbit, to generate polyclonal antibodies or, more preferably, by generating monoclonal antibodies. Alternatively, a clone encoding at least the Fab portion of the antibody may be obtained by screening Fab expression libraries (e.g. as described in Huse et al., 1989, Science 246:1275-1281) for clones of Fab fragments that bind the specific antigen or by screening antibody libraries (See, e.g. Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (e.g. WO 86/05807; WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulphide bond with an amino acid residue that does not contain a sulphydryl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem 253:6551), PCR based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g. humanised antibodies.

Once a nucleic acid encoding an antibody molecule has been obtained, the vector for the production of the antibody molecule may be produced by recombinant DNA technology using techniques well known in the art. Thus, methods for preparing these antibodies by expressing a nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an NKCC1 antibody.

The host cells used to express a recombinant NKCC1 antibody may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45(1): pp 101-5; Cockett et al., 1990, BioTechnology 8:2).

A variety of host-expression vector systems may be utilised to express an NKCC1 antibody molecule. Such host-expression systems represent vehicles by which the coding sequences of interest may be produced and subsequently purified, but also represent cells which may, when transformed or transfected with the appropriate nucleotide coding sequences, express the NKCC1 antibody in situ. These include but are not limited to microorganisms such as bacteria (e.g. E. coli, B. subtilis) transformed with recombinant bacteriophage DNA, plasmid DNA or cosmid DNA expression vectors containing antibody coding sequences; yeast (e.g. Saccharomyces, Pichia) transformed with recombinant yeast expression vectors containing antibody coding sequences; insect cell systems infected with recombinant virus expression vectors (e.g. baculovirus) containing the antibody coding sequences; plant cell systems infected with recombinant virus expression vectors (e.g. cauliflower mosaic virus, CaMV; tobacco mosaic virus, TMV) or transformed with recombinant plasmid expression vectors (e.g. Ti plasmid) containing antibody coding sequences; or mammalian cell systems (e.g. COS, CHO, BHK, HEK 293, 3T3 cells) harbouring recombinant expression constructs containing promoters derived from the genome of mammalian cells (e.g. metallothionein promoter) or from mammalian viruses (e.g. the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chen. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter). In mammalian host cells, a number of viral-based expression systems (e.g. an adenovirus expression system) may be utilised.

As discussed above, a host cell strain may be chosen which modulates the expression of the inserted sequences, or modifies and processes the gene product in the specific fashion desired. Such modifications (e.g. glycosylation) and processing (e.g. cleavage) of protein products may be important for the activity of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable marker (e.g. neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, “The use of vectors based on gene amplification for the expression of cloned genes in mammalian cells in DNA cloning”, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the NKCC1 antibody has been recombinantly expressed, it may be purified by any method known in the art for purification of an antibody molecule, for example, by chromatography (e.g. ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilising an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

In a preferred embodiment, NKCC1 antibodies are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In, and ⁹⁹Tc.

In one embodiment, the invention also provides diagnostic kits, comprising an NKCC1 capture reagent, e.g. an antibody. In addition, such a kit may optionally comprise one or more of the following:
(1) instructions for using the capture reagent for diagnosis, prognosis, therapeutic monitoring or any combination of these applications;
(2) a labelled binding partner to the capture reagent;
(3) a solid phase (such as a reagent strip) upon which the capture reagent is immobilised; and
(4) a label or insert indicating regulatory approval for diagnostic, prognostic or therapeutic use or any combination thereof.

If no labelled binding partner to the capture reagent is provided, the anti-NKCC1 capture reagent itself can be labelled with a detectable marker, e.g. a chemiluminescent, enzymatic, fluorescent, or radioactive moiety.

NKCC1 antibodies can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumour necrosis factor, α-interferon, β-interferon, nerve growth factor, platelet derived growth factor, tissue plasminogen activator, a thrombotic agent or an anti-angiogenic agent, e.g. angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor. Other therapeutic moieties may include radionuclides such as ¹¹¹In and ⁹⁰Y; antibiotics, e.g. calicheamicin; or drugs such as but not limited to, alkylphosphocholines, topoisomerase I inhibitors, taxoids and suramin.

Techniques for conjugating such therapeutic moiety to antibodies are well known, see, e.g. Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982) and Dubowchik et al., 1999, Pharmacology and Therapeutics, 83, 67-123.

Alternatively, an antibody can be conjugated to a second antibody to form an antibody heteroconjugate as described in U.S. Pat. No. 4,676,980.

An antibody with or without a therapeutic moiety conjugated to it can be used as a therapeutic that is administered alone or in combination with cytotoxic factor(s) and/or cytokine(s).

A further aspect of the invention provides methods of screening for active agents that modulate (e.g. upregulate or downregulate) a characteristic of, e.g. the expression or the enzymatic or binding activity of an NKCC1 polypeptide. The present invention also provides assays for use in drug discovery in order to identify or verify the efficacy of agents for treatment or prevention of breast, lung and/or pancreatic cancer. Candidate agents can be assayed for their ability to modulate levels of a polypeptide as defined herein in a subject having breast, lung and/or pancreatic cancer. Agents able to modulate levels of an NKCC1 polypeptide in a subject having breast, lung and/or pancreatic cancer towards levels found in subjects free from breast, lung and/or pancreatic cancer or to produce similar changes in experimental animal models of breast, lung and/or pancreatic cancer can be used as lead agents for further drug discovery, or used therapeutically. Expression of an NKCC1 polypeptide can be assayed by, for example, immunoassays, gel electrophoresis followed by visualisation, detection of activity, or any other method taught herein or known to those skilled in the art. Such assays can be used to screen candidate agents, in clinical monitoring or in drug development, where abundance of an NKCC1 polypeptide can serve as a surrogate marker for clinical disease.

Therefore, the present invention provides methods for identifying active agents that bind to an NKCC1 polypeptide or have a modulatory effect (e.g. stimulatory, inhibitory, up-regulatory or down-regulatory) effect on the expression or activity of an NKCC1 polypeptide. Examples of agents, include, but are not limited to, nucleic acids (e.g. DNA and RNA), carbohydrates, lipids, proteins, peptides, peptidomimetics, agonists, antagonists, small molecules and other drugs. Candidate agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Ian, 1997, Anticancer Drug Des. 12:145; U.S. Pat. No. 5,738,996; and U.S. Pat. No. 5,807,683).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of agents may be presented, e.g. in solution (e.g. Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. No. 5,571,698; U.S. Pat. No. 5,403,484; and U.S. Pat. No. 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

In one embodiment, agents that interact with (i.e. bind to) an NKCC1 polypeptide are identified in a cell-based assay system. In accordance with this embodiment, cells expressing an NKCC1 polypeptide are contacted with a candidate agent or a control agent and the ability of the candidate agent to interact with the polypeptide is determined. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express the NKCC1 polypeptide endogenously or be genetically engineered to express said polypeptide. In some embodiments, the NKCC1 polypeptide or the candidate agent is labelled, for example with a radioactive label (such as ³²P, ³⁵S or ¹²⁵I or a fluorescent label (such as fluorescein isothiocyanate, rhodamine, phycoerythrin, phycocyanin, allophycocyanin, o-phthaldehyde or fluorescamine) to enable detection of an interaction between a polypeptide and a candidate agent. The ability of the candidate agent to interact directly or indirectly with the NKCC1 polypeptide can be determined by methods known to those of skill in the art. For example, the interaction between a candidate agent and a polypeptide can be determined by flow cytometry, a scintillation assay, immunoprecipitation or western blot analysis.

In another embodiment, agents that interact with (i.e. bind to) an NKCC1 polypeptide are identified in a cell-free assay system. In accordance with this embodiment, a native or recombinant NKCC1 polypeptide is contacted with a candidate agent or a control agent and the ability of the candidate agent to interact with the polypeptide is determined. If desired, this assay may be used to screen a plurality (e.g. a library) of candidate agents. Preferably, the polypeptide is first immobilised, by, for example, contacting the polypeptide with an immobilised antibody which specifically recognises and binds it, or by contacting a purified preparation of polypeptide with a surface designed to bind proteins. The polypeptide may be partially or completely purified (e.g. partially or completely free of other polypeptides) or part of a cell lysate. Further, the polypeptide may be a fusion protein comprising the NKCC1 polypeptide or a biologically active portion thereof and a domain such as glutathionine-S-transferase. Alternatively, the polypeptide can be biotinylated using techniques well known to those of skill in the art (e.g. biotinylation kit, Pierce Chemicals; Rockford, Ill.). The ability of the candidate agent to interact with the polypeptide can be can be determined by methods known to those of skill in the art.

In another embodiment, a cell-based assay system is used to identify active agents that bind to or modulate the activity of a protein, such as an enzyme, or a biologically active portion thereof, which is responsible for the production or degradation of the NKCC1 polypeptide or is responsible for the post-translational modification of the polypeptide. In a primary screen, a plurality (e.g. a library) of agents are contacted with cells that naturally or recombinantly express: (i) an NKCC1 polypeptide; and (ii) a protein that is responsible for processing of the NKCC1 polypeptide in order to identify compounds that modulate the production, degradation, or post-translational modification of the polypeptide. If desired, active agents identified in the primary screen can then be assayed in a secondary screen against cells naturally or recombinantly expressing the specific polypeptide of interest. The ability of the candidate agent to modulate the production, degradation or post-translational modification of an NKCC1 polypeptide can be determined by methods known to those of skill in the art, including without limitation, flow cytometry, a scintillation assay, immunoprecipitation and Western blot analysis.

In another embodiment, agents that competitively interact with (i.e. competitively bind to) an NKCC1 polypeptide are identified in a competitive binding assay. In accordance with this embodiment, cells expressing the polypeptide are contacted with a candidate agent and an agent known to interact with the polypeptide; the ability of the candidate agent to competitively interact with the polypeptide is then determined. Alternatively, agents that competitively interact with (i.e. competitively bind to) a polypeptide are identified in a cell-free assay system by contacting the polypeptide with a candidate agent and an agent known to interact with the polypeptide. As stated above, the ability of the candidate agent to interact with a polypeptide for use in the invention can be determined by methods known to those of skill in the art. These assays, whether cell-based or cell-free, can be used to screen a plurality (e.g. a library) of candidate agents.

In another embodiment, active agents that modulate (i.e. upregulate or downregulate) the expression of an NKCC1 polypeptide or an NKCC1 nucleic acid are identified by contacting cells (e.g. cells of prokaryotic origin or eukaryotic origin) expressing the polypeptide or the nucleic acid with a candidate agent or a control agent (e.g. phosphate buffered saline (PBS)) and determining the expression of the polypeptide or the nucleic acid encoding the polypeptide. The level of expression of an NKCC1 polypeptide or NKCC1 nucleic acid in the presence of the candidate agent is compared to the level of expression of the polypeptide or nucleic acid in the absence of the candidate agent (e.g. in the presence of a control agent). The candidate agent can then be identified as a modulator of the expression of the polypeptide based on this comparison. For example, when expression of the polypeptide or nucleic acid is significantly greater in the presence of the candidate agent than in its absence, the candidate agent is identified as a stimulator of expression of the polypeptide or nucleic acid. Alternatively, when expression of the polypeptide or nucleic acid is significantly less in the presence of the candidate agent than in its absence, the candidate agent is identified as an inhibitor of the expression of the polypeptide or nucleic acid. The level of expression of an NKCC1 polypeptide, or the nucleic acid, can be determined by methods known to those of skill in the art based on the present description. For example, DNA or mRNA expression can be assessed by Northern blot analysis or RT-PCR, and protein levels can be assessed by Western blot analysis.

In another embodiment, active agents that modulate the activity of an NKCC1 polypeptide are identified by contacting a preparation containing the polypeptide, or cells (e.g. prokaryotic or eukaryotic cells) expressing the polypeptide with a candidate agent or a control agent and determining the ability of the candidate agent to modulate (e.g. stimulate or inhibit) the activity of the polypeptide. The activity of an NKCC1 polypeptide can be assessed by detecting its effect on a “downstream effector” for example, but without limitation, induction of a cellular signal transduction pathway of the polypeptide (e.g. intracellular Ca²⁺, diacylglycerol, IP₃, etc.), detecting catalytic or enzymatic activity of the target on a suitable substrate, detecting the induction of a reporter gene (e.g. a regulatory element that is responsive to an NKCC1 polypeptide and is operably linked to a nucleic acid encoding a detectable marker, e.g. luciferase), or detecting a cellular response, for example, cellular differentiation, or cell proliferation as the case may be, based on the present description, techniques known to those of skill in the art can be used for measuring these activities (see, e.g. U.S. Pat. No. 5,401,639). The candidate agent can then be identified as a modulator of the activity of an NKCC1 polypeptide by comparing the effects of the candidate agent to the control agent. Suitable control agents include phosphate buffered saline (PBS) and normal saline (NS).

In another embodiment, active agents that modulate the expression, activity or both the expression and activity of an NKCC1 polypeptide are identified in an animal model. Examples of suitable animals include, but are not limited to, mice, rats, rabbits, monkeys, guinea pigs, dogs and cats. Preferably, the animal used represents a model of breast, pancreatic and/or lung cancer. In accordance with this embodiment, the candidate agent or a control agent is administered (e.g. orally, rectally or parenterally such as intraperitoneally or intravenously) to a suitable animal and the effect on the expression, activity or both expression and activity of the polypeptide is determined. Changes in the expression of a polypeptide can be assessed by any suitable method described above, based on the present description. Alternatively, agents may be identified by monitoring the effect of their administration on symptoms associated with the disease or condition to be treated (e.g. to ameliorate symptoms or to delay onset or slow the progression of the disease). Therefore in a further embodiment, agents that reduce the severity of one or more symptoms associated with the disease or that slow the progression of the disease in a group of mammals treated with the candidate agent, compared to a untreated group of mammals are identified as potential active agents for the treatment of the disease. Techniques known to physicians familiar with breast, pancreatic and/or lung cancer can be used to determine whether a candidate agent has altered one or more symptoms associated with said diseases. For example, a candidate agent that reduces tumour burden in a subject having breast, pancreatic and/or lung cancer will be beneficial for treating breast, pancreatic and/or lung cancer patients.

In a particular embodiment, the methods of screening as described above may additionally comprise selecting an agent which modulates the expression or activity of said polypeptide or the expression of said nucleic acid molecule, for further testing or therapeutic or prophylactic use as an anti-breast, lung and/or pancreatic cancer agent.

In yet another embodiment, an NKCC1 polypeptide is used as a “bait protein” in a two-hybrid assay or three hybrid assay to identify other proteins that bind to or interact with the polypeptide (see, e.g. U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and WO 94/10300). As those skilled in the art will appreciate, such binding proteins are also likely to be involved in the propagation of signals by the NKCC1 polypeptides as, for example, upstream or downstream elements of a signalling pathway involving the NKCC1 polypeptides.

This invention further provides NKCC1 polypeptides, NKCC1 nucleic acids, NKCC1 antibodies, agents that modulate the expression or activity of a NKCC1 polypeptide, that interact with a NKCC1 polypeptide or that modulate the expression of a NKCC1 nucleic acid, including those identified by the above-described screening methods and uses thereof for treatments as described herein. Hereinafter, the agents, NKCC1 polypeptides, NKCC1 nucleic acids and NKCC1 antibodies are referred to as “active agents”. The term “treatment” includes either therapeutic or prophylactic therapy. When a reference is made herein to a method of treating or preventing a disease or condition using a particular active agent or combination of agents, it is to be understood that such a reference is intended to include the use of that active agent or combination of agents in the preparation of a medicament for the treatment of said disease or condition.

The invention also provides for treatment and/or prevention of breast, pancreatic and/or lung cancer by administration of an active agent.

In a further aspect, the present invention provides the use of an NKCC1 polypeptide in the production of a composition for the treatment or prophylaxis of breast, lung and/or pancreatic cancer, wherein the composition is a vaccine. The vaccine optionally comprises one or more suitable adjuvants. Examples of adjuvants well-known in the art include inorganic gels, such as aluminium hydroxide, and water-in-oil emulsions, such as incomplete Freund's adjuvant. Other useful adjuvants will be well known to the skilled person.

In yet further aspects, the present invention provides:
(a) the use of an NKCC1 polypeptide in the preparation of an immunogenic composition, preferably a vaccine;
(b) the use of such an immunogenic composition in inducing an immune response in a subject; and
(c) a method for the treatment or prophylaxis of breast, lung and/or pancreatic cancer in a subject, or of vaccinating a subject against breast, lung and/or pancreatic cancer which comprises the step of administering to the subject an effective amount of an NKCC1 polypeptide, preferably as a vaccine.

In one embodiment, one or more active agents are administered alone or in combination e.g. simultaneously, sequentially or separately, with one or more additional treatments or therapeutic agents for breast, lung and/or pancreatic cancer. Examples of such treatments include, surgery and radiation therapy. Examples of therapeutic compounds include but are not limited to cyclophosphamide (Cytoxan™); methotrexate (Methotrexate™); 5-fluorouracil (5-FU); paclitaxel (Taxol); docetaxel (Taxotere™); vincristine (Oncovin™); vinblastine (Velban™); vinorelbine (Navelbine™); doxorubicin (Adriamycin); tamoxifen (Nolvadex™); toremifene (Fareston™); megestrol acetate (Megace™); anastrozole (Arimidex™); goserelin (Zoladex™); anti-HER2 monoclonal antibody (Herceptin™); capecitabine (Xeloda™) and raloxifene hydrochloride (Evista™).

As discussed herein, active agents of the invention find use in the treatment or prophylaxis of breast, pancreatic and/or lung cancer. Thus, in a further aspect, the present invention provides a pharmaceutical composition comprising at least one active agent, optionally together with one or more pharmaceutically acceptable excipients, carriers or diluents. In one embodiment, the pharmaceutical composition is for use as a vaccine and so any additional components will be acceptable for vaccine use. In addition, the skilled person will appreciate that one or more suitable adjuvants may be added to such vaccine preparations.

The composition will usually be supplied as part of a sterile, pharmaceutical composition that will normally include a pharmaceutically acceptable carrier. This pharmaceutical composition may be in any suitable form (depending upon the desired method of administering it to a patient).

It may be provided in unit dosage form will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions).

Suitable excipients for tablets or hard gelatine capsules include lactose, maize starch or derivatives thereof, stearic acid or salts thereof.

Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions, oils (e.g. vegetable oils) may be used to provide oil-in-water or water in oil suspensions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base. Pharmaceutical compositions adapted for topical administration to the eye include eye drops wherein the active agent is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the active agent.

Pharmaceutical compositions adapted for administration by inhalation include fine particle dusts or mists which may be generated by means of various types of metered dose pressurised aerosols, nebulisers or insufflators.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solution which may contain anti-oxidants, buffers, bacteriostats and solutes which render the composition substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilised) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (polypeptides for use in the present invention may themselves be provided in the form of a pharmaceutically acceptable salt), buffers, coating agents or antioxidants.

Dosages of the active agents for use in the present invention can vary between wide limits, depending upon the stage of the breast, lung and/or pancreatic cancer, the age and condition of the individual to be treated, etc. and a physician will ultimately determine appropriate dosages to be used. This dosage may be repeated as often as appropriate. If side effects develop the amount and/or frequency of the dosage can be reduced, in accordance with normal clinical practice.

In view of the importance of NKCC1 in breast, pancreatic and/or lung cancer the following form additional aspects of the present invention:
i) a method for monitoring/assessing breast, pancreatic and/or lung cancer treatment in a patient, which comprises the step of determining the presence or absence and/or quantifying an NKCC1 polypeptide or an NKCC1 nucleic acid molecule in a biological sample obtained from said patient.
ii) methods of treating breast, pancreatic and/or lung cancer, comprising administering to a patient a therapeutically effective amount of an active agent that interacts with or modulates (e.g. upregulates or downregulates) or complements the expression or the biological activity (or both) of an NKCC1 polypeptide in patients having breast, pancreatic and/or lung cancer, in order to (a) prevent the onset or development of breast, lung and/or pancreatic cancer; (b) prevent the progression of breast, pancreatic and/or lung cancer; or (c) ameliorate the symptoms of breast, pancreatic and/or lung cancer.
iii) the use of an active agent, which interacts with, or modulates the expression or activity of an NKCC1 polypeptide in the preparation of a composition for the treatment of breast, lung and/or pancreatic cancer.
iv) a method for the prophylaxis and/or treatment of breast, lung and/or pancreatic cancer in a subject, which comprises administering to said subject a therapeutically effective amount of an antibody which binds to at least one NKCC1 polypeptide.
v) the use of an antibody which binds to at least one NKCC1 polypeptide in the preparation of a composition for use in the prophylaxis and/or treatment of breast, lung and/or pancreatic cancer.

Preferred features of each aspect of the invention are as for each of the other aspects mutatis mutandis.

The contents of each reference, patent and patent application cited in this application is hereby incorporated by reference in its entirety.

The invention will now be described with reference to the following examples, which should not in any way be construed as limiting the scope of the present invention. The examples refer to the figures in which:

FIG. 1: shows the nucleotide and amino acid sequences of NKCC1 (GenBank accession: U30246; SwissProt accession: P55011). The tandem spectra used to identify NKCC1 in breast and pancreatic cancer cell line membrane preparations are shown in bold, italicised, and underlined. Masses assigned to NKCC1 are shown in bold and italicised (see below).

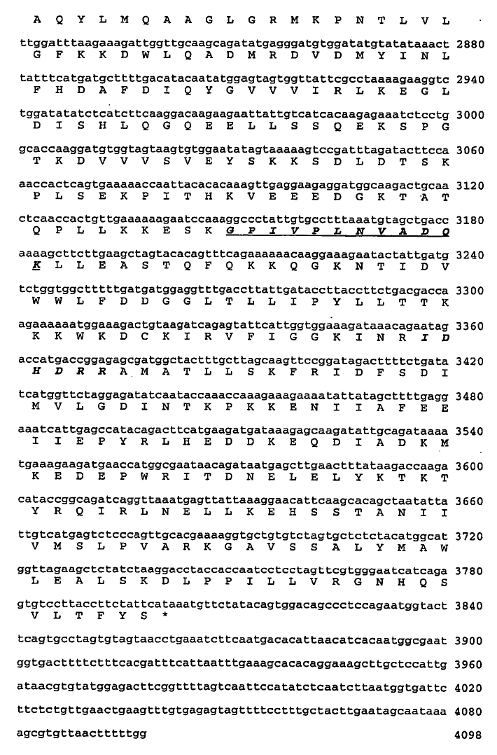

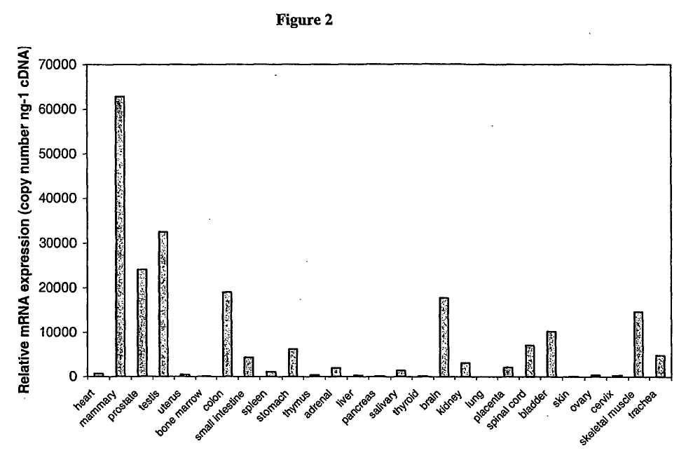

FIG. 2: shows the normal tissue distribution of NKCC1 mRNA. Levels of mRNA in normal tissues were quantified by real time RT-PCR. mRNA levels are expressed as the number of copies ng⁻¹ cDNA.

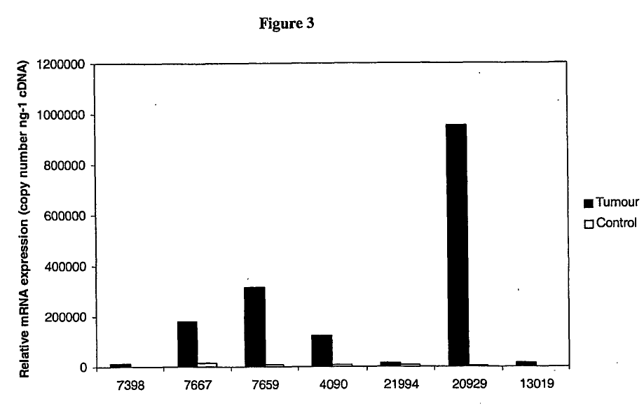

FIG. 3: shows the expression of NKCC1 mRNA in normal and breast cancer tissues. Levels of NKCC1 mRNA in donor matched normal and adjacent tumour tissues were measured by real time RT-PCR. mRNA levels are expressed as the number of copies ng⁻¹ cDNA.

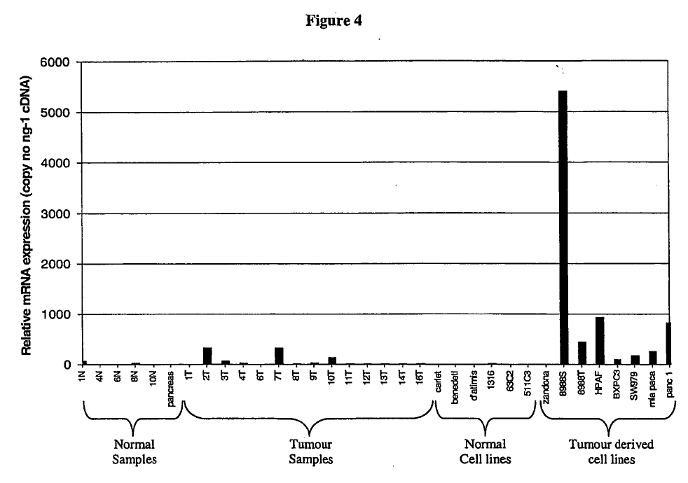

FIG. 4: shows the expression of NKCC1 mRNA in normal pancreatic tissues, pancreatic cancer tissues, and normal and tumour-derived pancreatic cell lines. Levels of NKCC1 mRNA were measured by real time RT-PCR. mRNA levels are expressed as the number of copies ng⁻¹ cDNA.

### EXAMPLE 1

### Identification of NKCC1

NKCC1 was Isolated from Breast Cancer (MDA MB 468/BT474) and Pancreatic (HPAFII) Cell Lines.

Cells were grown to confluence in 15 cm² cell culture dishes before fractionation. Before harvest and extraction, the cells (approx. 2×10⁹ cells) were washed three times with PBS-CM. Cells were scraped from culture dishes in ice-cold PBS-CM (5 ml per 2×10⁸ cells) using a plastic cell lifter. The cells were then centrifuged at 1000×g for 5 min at +4° C. The supernatant was removed and the cells were resuspended in 10 ml of homogenisation buffer (250 mM Sucrose in 10 mM HEPES, 1 mM EDTA 1 nM Vanadate, 0.02% Azide) followed by centrifugation at 1000×g for 5 min at +4° C. The supernatant was removed. The cell pellet was then resuspended in 5× packed cell volume with homogenisation buffer plus protease inhibitors (Sigma). A ball bearing homogeniser (BBH) (8.002 mm ball) was chilled and rinsed with homogenisation buffer. The cell suspension was taken up in a 2 ml syringe and attached to one side of the BBH. Another syringe was attached to the other side of the BBH. The cell mixture was fed through the chamber up to five times. Breakage of the cells was monitored using a microscope and when the cells were sufficiently lysed the resulting mixture was centrifuged at 1000×g for 5 min at +4° C.

The resulting supernatant (PNS) was retained and 1 ml of homogenisation buffer was added to the nuclear pellet followed by centrifugation at 1000×g for 5 min. The latter two fractions were pooled and centrifuged at 3000×g for 10 min at +4° C. The 3000×g supernatant was layered onto a 2 ml 60% sucrose cushion in SW40 or SW60 tube and centrifuged at 100 000×g for 45 min with slow acceleration and deceleration. The crude plasma membrane was evident as a discrete layer on top of the sucrose cushion. The upper layer was removed (cytosol) and the plasma membrane was collected using a pasteur pipette. The % sucrose of crude plasma membrane fraction was determined using a refractometer. The membrane preparation was diluted with HEPES buffer to reduce the sucrose content to below 15%. The crude plasma membrane preparation was layered on preformed 15 to 60% sucrose gradient in SW40 tube and spun at 100 000×g for 17 h with slow acceleration and deceleration.

The sucrose gradient was fractionated using the gradient unloader (speed 0.5, distance 2.5, fractions 35). The protein content of the fractions was measured and 10 micrograms of protein was run on a 4-20% acrylamide 1D gel (Novex) and subject to western blotting with antibodies to Transferrin Receptor, Oxidoreductase II and Calnexin.

Plasma membrane fractions that had transferrin immunoreactivity but no oxidoreductase II or calnexin immunoreactivity were identified. These sucrose fractions were pooled and diluted at least four times with 10 mM HEPES, 1 mM EDTA 1 mM Vanadate, 0.02% Azide. The diluted sucrose fraction was added to a SW40 or SW60 tube and centrifuged at 100 000×g for 45 min with slow acceleration and deceleration. The supernatant was removed from the membrane pellet and the pellet washed three times with PBS-CM. The membrane pellet was solubulized in 2% SDS in 63 mM TrisHCl, pH 7.4. A protein assay was performed followed by the addition of mercaptoethanol (2% final), glycerol (10%) and bromopheneol blue (0.0025% final). The extracted protein sample was finally solubilized in 1D lysis buffer to a final protein concentration of microgram/microlitre and the proteins separated by 1D PAGE.

Mass Spectrometry

Proteins excised from the 1D gel were digested with trypsin and analysed by MALDI-TOF-MS (Voyager STR, Applied Biosystems) using a 337 nm wavelength laser for desorption and the reflectron mode of analysis. Selected masses for NKCC1 were further characterised by tandem mass spectrometry using a QTOF-MS equipped with a nanospray ion source, (Micromass UK Ltd.). Prior to MALDI analysis the samples were desalted and concentrated using C18 Zip Tips™ (Millipore). Samples for tandem mass spectrometry (MS) were purified using a nano LC system (LC Packings, Amsterdam, The Netherlands) incorporating C18 SPE material.

Using the SEQUEST search program (Eng et al., 1994, J. Am. Soc. Mass Spectrom. 5:976 989), uninterpreted tandem mass spectra of tryptic digest peptides were searched against a database of public domain proteins constructed of protein entries in the non-redundant database held by the National Centre for Biotechnology Information (NCBI). This database is accessible at http://www.ncbi.nlm.nih.gov/ and also constructed of Expressed Sequence Tags entries (http://www.ncbi.nlm.nih.gov/dbEST/index.html). As a result of sequence database searching tandem amino acid sequences were found to match a SwissProt accession number: P55011 (Bumetanide-sensitive sodium-(potassium)-chloride cotransporter 1), see FIG. 1 (SEQ ID NO: 1), matched tandem sequences are shown in bold.

Additionally, sequences were identified using peptide mass data derived from mass spectrometer analysis and the MOWSE database search procedure. Peptide mass information can provide a ‘fingerprint’ signature sufficiently discriminating to allow for the unique and rapid identification of unknown sample proteins, independent of other analytical methods such as protein sequence analysis. Practical experience has shown that sample proteins can be uniquely identified using as few as 3-4 experimentally determined peptide masses when screened against a fragment database derived from over 50,000 proteins (D. J. C. Pappin, P. Hojrup and A. J. Bleasby ‘Rapid Identification of Proteins by Peptide-Mass Fingerprinting’. Current Biology (1993), vol 3, 327-332. and http://www.hgmp.mrc.ac.uk/Bioinformatics/Webapp/mowse/). The version of the code used for a MOWSE database search had the following modifications: the size of the parent protein is not included in the calculation such that large proteins such as titin no longer bias the score; instead the theoretical frequency of a peptide of mass (x) is estimated using the mass (x) of the peptide and the mean mass of an amino acid whilst allowing for a probability of 0.2 for a missed internal cleavage (by trypsin) and 0.1 for the probability of the occurrence of a proteolytic cleavage site.

The score assigned to a match on a peptide mass (x) is the logarithm of the probability of finding such a match at random and is inversely proportional to the frequency of fragments of that mass.

The score is thus calculated using the linear regression formula found by plotting the score for a match on peptide mass (x) against the mass (x). Two mass matches to predicted trypsin fragments were identified in this manner for NKCC1, which also matched to P55011, see FIG. 1 (SEQ ID NO: 1), the sequences which were identified through mass matching are shown in italics and underlined. The accuracy of this method was 20 ppm.

### EXAMPLE 2

### Expression of NKCC1 mRNA in Human Tissues

Real time quantitative RT-PCR was used (Heid, C. A., et al., Genome Res. 6, 986-994 (1996); Morrison, T. B., et al., Biotechniques 24, 954-958 (1998)) to analyse the distribution of NKCC1 mRNA in normal human tissues (FIG. 2), donor matched tumour and adjacent normal tissues from seven breast cancer patients, (FIG. 3), normal and tumour-derived pancreatic cell lines and tissue specimens (FIG. 4).

Quantification of NKCC1 mRNA by RT-PCR

Real-time RT-PCR was used to quantitatively measure NKCC1 expression in normal human tissue mRNAs (Clontech), donor matched tumour and adjacent normal tissues from breast cancer patients, normal and tumour-derived pancreatic cell lines and tissue specimens. Ethical approval for the normal and tumour tissue samples was obtained at surgery (University of Oxford, UK). The primers used for PCR were as follows:

(SEQ ID NO: 3)

sense, 5′ cacctactacctgcgcaccttc 3′,

(SEQ ID NO: 4)

antisense, 5′ gaccacagcatctctggttgga 3′,

Reactions contained 5 ng cDNA (prepared using Superscript first strand synthesis for RT-PCR kit (Life Technologies)), SYBR green sequence detection reagents (PE Biosystems), sense and antisense primers, and were assayed on an ABI7700 sequence detection system (PE Biosystems). The PCR conditions were 1 cycle at 50° C. for 2 min, 1 cycle at 95° C. for 10 min, and 40 cycles of 95° C. for 15 sec, 65° C. for 1 min. The accumulation of PCR product was measured in real time as the increase in SYBR green fluorescence, and the data were analysed using the Sequence Detector program v1.6.3 (PE Biosystems). Standard curves relating initial template copy number to fluorescence and amplification cycle were generated using the amplified PCR product as a template, and were used to calculate NKCC1 copy number in each sample.

Overall the distribution of NKCC1 mRNA was low in normal tissues, with the highest levels of mRNA expression seen in mammary, prostate, testis and brain tissues (FIG. 2).

The expression of NKCC1 mRNA in clinical breast carcinoma tissues was compared with the matched adjacent normal tissue from 7 breast cancer patients (FIG. 3). NKCC1 expression was increased in all of the tumour samples, relative to their matched control tissue, with six of the seven samples showing a greater than 6-fold increase in expression.

NKCC1 mRNA expression was also analysed in normal and tumour pancreatic tissues and cell lines (FIG. 4). NKCC1 expression was found to be significantly higher in the pancreatic tumour tissue (mean=69 copies ng⁻¹ cDNA) and tumour-derived cell lines (mean=1013 copies ng⁻¹, cDNA) than in the corresponding normal pancreatic tissues and cell lines (mean=16 and 5 copies per ng⁻¹ cDNA respectively).

### EXAMPLE 3

### Immunohistochemical Analysis of NKCC1 in Breast, Lung and Pancreatic Cancer Tissues

To further illustrate the involvement of NKCC1 in breast cancer, immunohistochemistry with a specific anti-NKCC1 antibody was used to investigate NKCC1 protein expression in sections of multiple donor breast ductal carcinoma tissues and in lung adenocarcinoma and pancreatic cancer tissues.

Antibody Generation.

The anti-NKCC1 polyclonal antibody was raised in guinea pigs immunised with 2 specific peptides (Immune Systems Limited, UK). Peptide sequences were chosen for synthesis based on plots of hydrophobicity, antigenicity, surface probability, and weak homology to other known protein family members. Peptides were synthesised using Fmoc chemistry with a cysteine residue added to the end of each to enable specific thiol-reactive coupling of Keyhole Limpet Haemocyanin prior to immunisation. The peptides used were; SKKPKGFFGYKC (SEQ ID NO: 5) and SGESEPAKGSEEAKGC (SEQ ID NO: 6). The antibodies were affinity purified using columns of the above immobilised peptides.

NKCC1 Immunohistochemistry in Breast Carcinoma Tissue

Immunohistochemical analysis was carried out on formalin-fixed paraffin-embedded tissue microarrays containing mm sections of breast carcinoma tissue from 55 donors as well as 20 sections of various normal tissues (Clinomics Laboratories Inc., 165 Tor Court, Pittsfield, Mass. 01201). Slides were deparafinised by 2×5 min washes in xylene then rehydrated through successive graded ethanol solutions and washed for 5 min in PBS. Antigen retrieval was achieved by immersing the slides in 0.01M citrate buffer (pH 6) and microwaving for 10 min at full power (950 W). In addition, detection with the antibody was improved by protease treatment of the tissue with Autozyme (AbCam) for 10 min at room temperature. The tissue was blocked in 10% donkey serum/PBS for 1 h before addition of 1.5 μg/ml primary polyclonal antibody (in 2.5% donkey serum/PBS). Following 3 washes in PBS the tissue sections were incubated with biotin-conjugated secondary antibodies (Biotin-SP-conjugated AffiniPure Donkey anti-guinea pig, Jackson ImmunoResearch) diluted at 1:200 (2.5 μg/ml in 2.5% donkey serum/PBS) for 1 h. Slides were washed 3 times in PBS and the tissue incubated with Streptavidin-HRP (Jackson ImmunoResearch) diluted 1:100 (5 μg/ml in 2.5% donkey serum/PBS), followed by 3×5 min washes in PBS. Antibody signal was detected using DAB substrate solution (Dako Ltd.) according to the manufacturers' instructions. An adjacent tissue array was counterstained for hematoxylin and eosin (Dako Ltd.) and images were captured by a digital camera attached to a light microscope.

NKCC1 immunostaining was seen in breast cancer tissue and it was clearly apparent that NKCC1 is specifically and highly expressed in the ductal carcinoma cells of the breast cancer tissue (compare with adjacent breast tissue). In total 55 breast cancer donor tissues were examined for NKCC1 immunoreactivity. Of these 7 demonstrated very high NKCC1 staining, 30 exhibited high/moderate staining, 13 exhibited weak staining, and 5 showed no staining in the carcinoma cells.

NKCC1 Immunohistochemistry in Lung and Pancreatic Cancer Tissues

Immunohistochemical analysis was carried out on formalin-fixed paraffin embedded tissue microarrays containing 1 mm sections of carcinoma tissues from a wide range of donors (obtained from Clinomics Laboratories Inc., 165 Tor Court, Pittsfield, Mass. 01201). Slides were deparafinized by 2×5 min washes in xylene then rehydrated through successive graded ethanol solutions and washed for 5 min in PBS. Antigen retrieval was achieved by immersing the slides in 0.01M citrate buffer (pH 6) and microwaving for 10 min at full power (950 W) then treating the tissue with pepsin (1 mg/ml) for 1 min at room temperature at pH 2. The tissue was blocked in 10% donkey serum/PBS for 1 h before addition of 1 μg/ml primary anti-NKCC1 polyclonal antibody (in 2.5% donkey serum) for 1 h. Following three washes in PBS the tissue sections were incubated with biotin-conjugated secondary antibodies (Biotin-SP-conjugated AffiniPure Donkey anti-guinea pig IgG, Jackson ImmunoResearch) diluted at 1:200 (2.5 μg/ml in 2.5% donkey serum/PBS) for 1 h. Slides were washed 3 times in PBS and the tissue incubated with Streptavidin-HRP (Jackson ImmunoResearch) diluted 1:100 (5 μg/ml in 2.5% donkey serum/PBS) for 1 h, followed by 3×5 min washes in PBS. Antibody signal was detected using DAB substrate solution (Dako Ltd.) according to the manufacturer's instructions.

Increased staining of the sections for NKCC1 was seen in both lung and pancreatic tissue sections compared to adjacent control sections.

### EXAMPLE 4

### Cellular Localisation of NKCC1 in Recombinant Cell Lines

Fluorescent immunocytochemistry was used to assess the cellular localisation of NKCC1 in recombinant cell lines.

The full-length open reading frame of human NKCC1 (SEQ ID NO: 2) was PCR cloned into the plasmid vector pcDNA3.1 (Invitrogen). HEK293 cells and CH0-K1 cells were stably transfected with NKCC1.pcDNA3.1 plasmid (Invitrogen) using transfection reagent GeneJuice™ (Novagen) according to the manufacturer's instructions. A pool of HEK293 cells expressing full-length NKCC1 was selected for growth in antibiotic-containing medium (0.2 mg/ml G418). CHO-K1 cells expressing full-length NKCC1 were dilution cloned and selected for growth in antibiotic-containing medium (0.2 mg/ml G418). Two CHO-K1 clones, 4D8 and 3C5, were selected for further assessment.

Transfected and non-transfected HEK293 cells and CHO-K1 cells were seeded onto 8-well chamber slides (Nalge Nunc) at a density of 6×10⁴ cells per well. After 24 h of incubation at 37° C. and 5% CO₂ the media was removed from the slides and the plastic housing of the chambers was also removed. The slides were washed once in PBS in a Coplin jar for five minutes. Excess PBS was removed from the slides and then they were placed in 100% acetone for 5 min. Following this incubation the slides were air dried briefly and then laid flat in a humidifier chamber and incubated for 1 h with 500 μl of primary antibody appropriately diluted in PBS 1% (w/v) BSA (2 μg/ml of guinea-pig anti-NKCC1 polyclonal antibody raised against peptide SEQ. ID NO: 5 (Immune Systems Limited, UK) or 2 μg/ml guinea-pig gamma globulin (Sigma)). Slides were then washed three times for 5 min per wash in PBS. The slides were-incubated for 1 h in a humidifier chamber with 500 μl of biotin-SP-conjugated AffiniPure donkey anti-guinea pig antibody, (Jackson ImmunoResearch) diluted 1:200 in PBS 1% (w/v) BSA. Following this incubation the slides were washed three times for 5 min per wash in PBS and then incubated for 1 h in a humidifier chamber with 500 μl of Cy3-conjugated-Extravidin (Sigma) diluted 1:700 in PBS 1% (w/v) BSA. Following this incubation the slides were washed four times for 5 min per wash in PBS. Slides were then incubated for 30 sec with 2 μg/nm BisBenzimide (Sigma) and then washed for 2 min in PBS. Slides were mounted with a coverslip in fluorescent enhancing media (Dako Ltd.) and then images were captured by digital camera DC300F attached to a DMIRE2 fluorescence microscope (Leica Microsystems (UK) Ltd.)

The resulting images clearly showed that NKCC1 was highly expressed on the plasma membrane of NKCC1-transfected HEK293 and CHO cells. This staining is highly specific to the NKCC1 transfected cells since staining of non-transfected HEK293 and CHO-K1 cells with anti-NKCC1 polyclonal antibody revealed very low levels of fluorescence. Control guinea-pig IgG staining of NKCC1 transfected cells gave a very low level of background fluorescence.

Thus, NKCC1 shows a restricted pattern of expression in normal human tissues, and is elevated in breast cancer tissues, lung cancer tissues, pancreatic cancer tissues and cancer cell lines. Immunocytochemistry in cell lines transfected with NKCC1 demonstrated a clear membrane association, indicating that NKCC1 has potential in an immunotherapeutic based approach to the treatment and/or prophylaxis of breast, lung and/or pancreatic cancer.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims.

1. A method of screening for and/or diagnosis of breast, lung and/or pancreatic cancer in a subject and/or monitoring the effectiveness of breast, lung and/or pancreatic cancer therapy, which method comprises the step of detecting and/or quantifying in a biological sample obtained from said subject:
(i) an NKCC1 polypeptide which:
a) comprises or consists of the amino acid sequence shown in FIG. 1 (SEQ ID NO: 1);
b) is a derivative having one or more amino acid substitutions, modifications, deletions or insertions relative to the amino acid sequence shown in FIG. 1 (SEQ ID NO: 1) which retains the immunological or biological activity of NKCC1; or
c) is a fragment of a polypeptide having the sequence shown in FIG. 1 (SEQ ID NO: 1), which is at least ten amino acids long and has at least 70% sequence identity over the length of the fragment; and/or
(ii) a nucleic acid molecule which:
d) comprises or consists of the DNA sequence shown in FIG. 1 (SEQ ID NO:2) or its RNA equivalent;
e) is a sequence which codes for a polypeptide as defined in a), b) or c);
f) is a sequence which is complementary to the sequences of d) or e);
g) is a sequence which codes for the same polypeptide, as the sequences of d) or e);
h) is a sequence which shows substantial identity with any of those of d), e), f) or g), or
i) is a fragment of d), e), f), g) or h), which is at least 8 nucleotides in length.

2. The method of claim 1, wherein the level of said polypeptide or said nucleic acid is compared to a previously determined reference range or control.

3. The method according to claim 1, wherein the step of detecting comprises:
(a) contacting the sample with a capture reagent that is specific for a polypeptide as defined in claim 1(i); and
(b) detecting whether binding has occurred between the capture reagent and said polypeptide in the sample.

4. The method according to claim 3, wherein step (b) comprises detecting the captured polypeptide using a directly or indirectly labelled detection reagent.

5. The method according to claim 3, wherein the capture reagent is immobilised on a solid phase.

6. An antibody, functionally-active fragment, derivative or analogue thereof, that specifically binds to a polypeptide as defined in claim 1(i).

7. The method according to claim 1, wherein the NKCC1 polypeptide is detected and/or quantified using an antibody that specifically binds to the polypeptide.

8. An antibody according to claim 6, wherein the antibody is monoclonal, polyclonal, chimeric, bispecific, humanised or is conjugated to a detectable substance, therapeutic moiety, a second antibody or a fragment thereof, a cytotoxic agent or a cytokine.

9. A diagnostic kit comprising a capture reagent specific for an NKCC1 polypeptide as defined in claim 1(i), reagents and instructions for use.

10. A method for the prophylaxis and/or treatment of breast, lung and/or pancreatic cancer in a subject, which comprises administering to said subject a therapeutically effective amount of:
i) a polypeptide as defined in claim 1(i), or
ii) a nucleic acid molecule as defined in claim 1(ii).

11. A method for the prophylaxis and/or treatment of breast, lung and/or pancreatic cancer in a subject, which comprises administering to said subject a therapeutically effective amount of an antibody as defined in claim 6.

12. (canceled)

13. (canceled)

14. (canceled)

15. (canceled)

16. The method of claim 10, wherein the polypeptide and the nucleic acid molecule are prepared as a composition, and wherein the composition is a vaccine.

17. The method of claim 10, where the nucleic acid inhibits the expression of the polypeptide as defined in claim 1(i).

18. A method of screening for agents that interact with a polypeptide as defined in claim 1(i), said method comprising:
(a) contacting said polypeptide with a candidate agent; and
(b) determining whether or not the candidate agent interacts with said polypeptide.

19. The method according to claim 18, wherein the determination of interaction between the candidate agent and the polypeptide comprises quantitatively detecting binding of the candidate agent and said polypeptide.

20. A method of screening for anti-breast, pancreatic and/or lung cancer agents that modulate
i) the expression or activity of a polypeptide as defined in claim 1(i), or
ii) the expression of a nucleic acid molecule as defined in claim 1(ii), said method comprising:
a) comparing the expression or activity of said polypeptide or the expression of said nucleic acid molecule, in the presence of a candidate agent with the expression or activity of said polypeptide or the expression of said nucleic acid molecule, in the absence of the candidate agent or in the presence of a control agent; and
b) determining whether the candidate agent causes the expression or activity of said polypeptide or the expression of said nucleic acid molecule, to change.

21. The method of claim 20 wherein the expression or activity level of said polypeptide or the expression level of said nucleic acid molecule is compared with a predetermined reference range.

22. The method of claims 20 wherein step b) additionally comprises selecting an agent which modulates the expression or activity of said polypeptide or the expression of said nucleic acid molecule, for further testing or therapeutic or prophylactic use as an anti-breast, lung and/or pancreatic cancer agent.

23. An active agent identified by the method of claim 18 which interacts with said polypeptide or causes the expression or activity of said polypeptide or the expression of said nucleic acid molecule, to change.

24. (canceled)

25. An active agent which interacts with a polypeptide as defined in claim 1(i), which modulates the expression or activity of said polypeptide or which modulates the expression of a nucleic acid as defined in claim 1(ii), for use in the prophylaxis and/or treatment of breast, lung and/or pancreatic cancer.

26. A method of prophylaxis and/or treatment of breast, lung and/or pancreatic cancer, which comprises administering to said subject a therapeutically effective amount of an active agent which interacts with a polypeptide as defined in claim 1(i), which modulates the expression or activity of said polypeptide or which modulates the expression of a nucleic acid as defined in claim 1(ii).

27. An active agent identified by the method of claim 22 which interacts with said polypeptide or causes the expression or activity of said polypeptide or the expression of said nucleic acid molecule, to change.
